(19) United States
(12) Patent Application Publication
(10) Pub. No.: US 2016/0361414 A1
(43) Pub. Date: Dec. 15, 2016

(54) Methods of Treating and Preventing Minor Histocompatibility Antigen-Mismatched Grafts

(71) Applicant: National Jewish Health, Denver, CO (US)

(72) Inventor: Claudia Jakubzick, Boulder, CO (US)

(21) Appl. No.: 15/181,115

(22) Filed: Jun. 13, 2016

Related U.S. Application Data
(60) Provisional application No. 62/175,041, filed on Jun. 12, 2015.

(51) Int. Cl. A61K 39/395; A61K 45/06; C07K 16/28

(52) U.S. Cl. CPC A61K 39/3955; C07K 16/2815; C07K 16/2866; C07K 16/2839; C07K 16/2833; C07K 16/2851; A61K 45/06; C07K 2317/76

(57) ABSTRACT

Disclosed herein are methods of preventing and/or reducing minor histocompatibility antigen-mismatched grafts by depletion and/or inhibition of basic leucine zipper transcription factor ATF-like 3 (Batf3)-dependent antigen-presenting cells.

### CROSS-REFERENCE TO RELATED APPLICATION

This application claims the benefit of priority under 35 U.S.C. §119(e) to U.S. Provisional Patent Application No. 62/175,041, filed Jun. 12, 2015. The entire disclosure of U.S. Provisional Patent Application No. 62/175,041 is incorporated herein by reference.

### GOVERNMENT SUPPORT

This invention was made with government support under grant number R01 HL115334 received from the National Institutes of Health National Heart, Lung, and Blood Institute. The government has certain rights in the invention.

### FIELD OF THE INVENTION

The field of the present invention involves methods of treating, preventing and/or reducing minor histocompatibility antigen-mismatched grafts by depletion and/or inhibition of basic leucine zipper transcription factor ATF-like 3 (Batf3)-dependent antigen-presenting cells.

### BACKGROUND

In transplantation, a major obstacle for graft acceptance in major histocompatibility complex (MHC) matched individuals is the mismatch of minor histocompatibility antigens. Minor H antigens are peptides derived from polymorphic proteins that can be presented by antigen-presenting cells (APC) on MHC molecules. The APC subtype uniquely responsible for the rejection of minor antigen-mismatched grafts has not yet been identified.

Dendritic cells (DCs) are professional antigen-presenting cells with the capacity to initiate T cell-mediated immunity. In murine non-lymphoid and lymphoid tissues, there are two major forms of classical DCs: Batf3-dependent and Batf3-independent DCs (Hildner, K., et al. 2008. Batf3 deficiency reveals a critical role for CD8alpha+ dendritic cells in cytotoxic T cell immunity. Science 322: 1097-1100). These DCs are distinguished by their cell surface expression for CD103/CD8/XCR1 and CD11b/CD4/SIRPα (Sung, S. S., et al. 2006. A major lung CD 103 (alphaE)-beta7 integrin-positive epithelial dendritic cell population expressing Langerin and tight junction proteins. Journal of immunology 176: 2161-2172; Vremec, D., et al. 1992. The surface phenotype of dendritic cells purified from mouse thymus and spleen: investigation of the CD8 expression by a subpopulation of dendritic cells. The Journal of experimental medicine 176: 47-58; Desch, A. N., et al. 2011. CD103+ pulmonary dendritic cells preferentially acquire and present apoptotic cell-associated antigen. The Journal of experimental medicine 208: 1789-1797), as well as pattern recognition receptors, antigen presentation and processing, and particularly, transcription factors involved in their development (Batf3 or IRF4, respectively) (Hildner, K., et al.; Plantinga, M., et al. 2010. Origin and functional specializations of DC subsets in the lung. Eur J Immunol 40: 2112-2118; Kim, T. S., and T. J. Braciale. 2009. Respiratory dendritic cell subsets differ in their capacity to support the induction of virus-specific cytotoxic CD8+ T cell responses. PloS one 4: e4204; Tamura, T., P. et al. 2005. IFN regulatory factor-4 and -8 govern dendritic cell subset development and their functional diversity. Journal of immunology 174: 2573-2581; Quantz, M. A., et al. 2000. Does human leukocyte antigen matching influence the outcome of lung transplantation? An analysis of 3,549 lung transplantations. The Journal of heart and lung transplantation: the official publication of the International Society for Heart Transplantation 19: 473-479). Others as well as the inventors have demonstrated the selective ability of Batf3-dependent DCs but not Batf3-independent DCs to take up apoptotic cells (i.e. self), migrate to the draining lymph nodes (LNs) and there present exogenous cell-associated antigen peptides on MHC class I (i.e. cross-presentation) (Desch, A. N., et al. 2011; Contreras, V., et al. 2010. Existence of CD8alpha-like dendritic cells with a conserved functional specialization and a common molecular signature in distant mammalian species. Journal of immunology 185: 3313-3325; Iyoda, T., et al. 2002. The CD8+ dendritic cell subset selectively endocytoses dying cells in culture and in vivo. The Journal of experimental medicine 195: 1289-1302; Ferguson, T. A., et al. 2002. Uptake of apoptotic antigen-coupled cells by lymphoid dendritic cells and cross-priming of CD8(+) T cells produce active immune unresponsiveness. Journal of immunology 168: 5589-5595; den Haan, J. M., et al. 2000. CD8(+) but not CD8(−) dendritic cells cross-prime cytotoxic T cells in vivo. The Journal of experimental medicine 192: 1685-1696), which can then be recognized by cognate CD8⁺ T cells. Subsequently, depending on the activation status of antigen-presenting DCs, proliferating antigen-specific CD8⁺ T cells can be instructed to develop into cytotoxic T cells (i.e. cross-priming) (Kurts, C., et al. 2010. Cross-priming in health and disease. Nature reviews. Immunology 10: 403-414; Desch, A. N., et al. 2014. Dendritic cell subsets require cis-activation for cytotoxic CD8 T-cell induction. Nature communications 5: 4674). The induction of cytotoxic T cells by Batf3-dependent DCs has demarcated its beneficial roles in anti-viral and anti-tumor immunity (Hildner, K., et al.; Kim, T. S., et al.; Desch, A. N. et al. 2014; Edelson, B. T., et al. 2010. Peripheral CD103+ dendritic cells form a unified subset developmentally related to CD8alpha+ conventional dendritic cells. The Journal of experimental medicine 207: 823-836; Bedoui, S., et al. 2009. Cross-presentation of viral and self antigens by skin-derived CD103+ dendritic cells. Nature immunology 10: 488-495; GeurtsvanKessel, C. H., et al. 2008. Clearance of influenza virus from the lung depends on migratory langerin+CD11b—but not plasmacytoid dendritic cells. The Journal of experimental medicine 205: 1621-1634). However, detrimental roles for Batf3-dependent DCs have also been demonstrated in autoimmune diabetes (Ferris, S. T., et al. 2014. A minor subset of Batf3-dependent antigen-presenting cells in islets of Langerhans is essential for the development of autoimmune diabetes. Immunity 41: 657-669).

Due to the difficulties and inadequacies of conventional therapy for treating and reducing transplant complications and associated side effects, new therapeutic modalities are needed. As provided herein, the inventor demonstrates a new role for Batf3-dependent DCs in promoting the rejection of minor antigen-mismatched grafts, thus providing for methods of treating, preventing and reducing minor histocompatibility antigen mismatched grafts.

### SUMMARY OF THE INVENTION

One embodiment of the invention relates to a method of treating or preventing graft versus host disease in a subject in need thereof comprising administering to the subject a therapeutically effective amount of a pharmaceutical composition comprising an antibody against human antigen presenting cell (APC)-specific surface molecules, wherein the APC is a dendritic cell.

In one aspect, the APC-specific surface molecules are selected from the group consisting of cluster of differentiation 103 (CD103), cluster of differentiation 8 (CD8), chemokine (C Motif) Receptor 1 (XCR1) and combinations thereof.

In still another aspect, the antibody depletes or inhibits Batf3-dependent antigen presenting cells. In one aspect, the antibody is selected from the group consisting of cluster of differentiation 103 (CD103), cluster of differentiation 8 (CD8), chemokine (C Motif) Receptor 1 (XCR1) and combinations thereof.

Another embodiment of the invention relates to a method of treating or preventing rejection of minor antigen-mismatched grafts in a subject in need thereof, comprising administering to the subject a therapeutically effective amount of a pharmaceutical composition comprising an agent capable of depleting or inhibiting Batf3-dependent antigen presenting cells.

Another embodiment of the invention relates to a method of depleting or inhibiting Batf3-dependent antigen-presenting cells in a subject in need thereof comprising administering to the subject a pharmaceutical composition comprising an agent for inducing depletion or inhibition of Batf3-dependent cells.

In one aspect, the agent binds to Batf3-dependent cells.

In one aspect, the agent is an antibody.

In still another aspect, the subject is a transplant recipient.

In one aspect of any of the embodiments related to a method, the subject has or will receive an allograft. In one aspect, the subject is an MHC-matched individual to the allograft. In one aspect, the allograft is an organ, tissue or cells.

In one aspect of any of the embodiments related to a method, the pharmaceutical composition is administered concurrently, following or prior to a transplant of a graft.

In one aspect of any of the embodiments related to a method, the antibody is selected from the group consisting of C-type lectin domain family 9 (Clec9a), Cluster of Differentiation 1a (CD1a), DEC205, Cluster of Differentiation 1c (CD1c) and combinations thereof.

In one aspect of any of the embodiments related to a method, the method further comprises administering a toll-like receptor 7 (TLR7) inhibitor to the subject.

### BRIEF DESCRIPTION OF THE DRAWINGS

FIGS. 1A-1H show rejection of male-specific minor antigens requires Batf3-dependent DCs. (FIG. 1A) Gated CD8⁺ T cells display the recall of adoptively transferred (AT) CD45.1 male Va2⁺ OT-I T cells (gray gate) in WT male and female mice. (FIG. 1B) In vivo Cytotoxic T lymphocyte (CTL) assay, male OT-I cells were AT into WT male and female mice 1 day (d) prior to intranasal (i.n.) 2 μg OVA, which expands male antigen-specific T cells, 10 d later, mice were intravascular (i.v.) (1:1) carboxyfluorescein diacetate succinimyidyl ester (CFSE)-labeled male (CD45.2⁺) and female (CD45.1⁺) target cells. 3 d post target cell AT, cytotoxicity was assessed. (FIG. 1C-1E) Gated CD8 (FIG. 1C upper panels) or CD4 (FIG. 1C lower panels) T cells display the recall of AT CD45.1 male Va2⁺T cells (gray gate, OT-I FIG. 1C upper panels and OT-II FIG. 1C lower panels) in BL/6 WT and Batf3^{-/-} male and female recipient mice. Scatter plots display frequency of OT-I (FIG. 1D) and OT-II (FIG. 1E) T cells from total CD8 and CD4 T cells. (FIG. 1F) Gated CD8 (FIG. 1F upper panels) or CD4 (FIG. 1F lower panels) T cells display the recall of AT CD45.1⁺KJ1-26⁺ or CD90.1⁺CD44⁺male T cells (gray gate, CL4 FIG. 1F upper panels and DO11.10 FIG. 1F lower panels) from Balb/c WT and Batf3^{-/-} male and female recipient mice. Scatter plot displays frequency of CL4 (FIG. 1G) and DO11.10 (FIG. 1H) T cells from total CD8 and CD4 T cells. (FIGS. 1B-1H) Data is representative of three independent experiments.

FIGS. 2A-2C show rejection of mismatched complex minor antigens requires Batf3-dependent DCs using the experimental design as in FIG. 1A. Gated CD8 T cells display the recall of CD45.1⁺Va2⁺AT male (FIG. 2A upper panel) and female (FIG. 2A lower panel) OT-I T cells (gray gate) from 129SvEv/BL6 WT and Batf3^{-/-} male and female recipient mice. Scatter plots display frequency of OT-I T cells from total CD8 T cells (FIGS. 2B-2C). Data is representative of three independent experiments.

FIGS. 3A-3B show rejection of male skin grafts on female recipients requires Batf3-dependent DCs. Representative pictures show transplanted murine skin grafts from Balb/c Batf3^{-/-} male and female donors onto Balb/c WT and Batf3^{-/-} female recipients, day 60 (FIG. 3A). Survival graph (FIG. 3B) displays male Batf3^{-/-} skin onto WT female (darker colored diamond, 0/6) and Batf3^{-/-} female (darker colored circle, 6/6) recipients. Controls, survival graph of female Batf3^{-/-} skin onto WT female (lighter colored diamond, 5/6) and Batf3^{-/-} female (lighter colored circle, 6/6) recipients. Data represents two independent experiments.

FIGS. 4A-4B show TLR7 ligand activates Batf3-independent DCs to promote rejection of mismatched complex minor antigens, using experimental design as in FIG. 1A. Plots (FIG. 4A) display the recall of AT CD45.1 female C57BL/6 OT-I T cells (gray gate) from 129SvEv/BL6 WT and Batf3^{-/-} female mice immunized with sOVA +/− poly I:C (20 μg) or R848 (50 μg). Scatter plot (FIG. 4B) displays frequency of OT-I T cells from total CD8 T cells. Data is representative of three independent experiments.

FIGS. 5A-5D show non-lymphoid CD103⁺ and lymphoid CD8⁺ Batf3-dependent DCs selectively engulf apoptotic cells, whereas soluble OVA is acquired and trafficked by both Batf3-dependent and Batf3-independent migratory DCs. (FIG. 5A) Lung-draining LNs or spleen were isolated 24 h post i.n. or i.v. delivery of CFSE-labeled apoptotic cells. Non-lymphoid and lymphoid DCs were gated and plotted as CD103 or CD8 versus CFSE (to identify efferocytic DCs). Data represents four independent experiments. (FIG. 5B) C57BL/6 mice were inoculated with OVA-FITC. Lung-draining LNs (upper panels) and distal skin-draining LNs (as control, lower panels) were isolated 24 h later and analyzed by flow. OVA⁺ trafficking cells were identified as FITC⁺. Overlay shows OVA⁺ cells were migratory DCs CD11c⁺ MHCII^{hi} (darker color) from total live cells (lighter color). Top left plot shows that migratory DCs, Batf3-dependent CD103⁺ and Batf3-independent CD11b⁺ DCs, traffic OVA antigen to the lung-draining LNs. (FIG. 5C) Schematic diagram of experimental design for the induction of male- antigen rejection in female mice. (FIG. 5D) Using experimental design as described in schematic diagram in FIG. 1A. Single-cells from lung-draining LN were gated on CD8 T cells, scatter plot display the frequency of recalled female OT-I CD8 T cells from C57BL/6 WT and Batf3^{-/-} male and female recipient mice. Data is representative of three independent experiments.

FIGS. 6A and 6B shows the induction of male antigen-specific CTL of male skin grafts on female recipients requires Batf3-dependent DCs. (FIG. 6A) Allogeneic T cell rejection 9 h, 60 h and 120 h post AT of BL/6 lymphocytes into Balb/c WT and Batf3^{-/-} mice. (FIG. 6B) In vivo CTL assay. 30 days post male skin transplant onto Batf3^{-/-} or WT female mice, mice were i.v. injected with (1:1) CFSE-labeled male (CD45.2⁺) and female (CD45.1⁺) target cells. 3d post target cell transfer, cytotoxicity was assessed. Data represents 2 of the 4 mice per group.

### DETAILED DESCRIPTION OF THE INVENTION

The present invention generally relates to novel methods of preventing and/or reducing minor histocompatibility antigen-mismatched grafts by depletion and/or inhibition of Batf3-dependent antigen-presenting cells.

The clinical implications of identifying the critical APC for presentation of mismatched minor antigens in transplantation are immense and span both human leukocyte antigen (HLA)-matched and unmatched transplantation. To underscore the importance of minor antigen allo-immunity in current clinical transplantation; multiple organs are not entirely HLA-matched to recipients because outcomes are only modest (Opelz et al., 1999), trivial (Quantz et al., 2000), or not (Opelz et al., 1999) impacted compared to HLA-mismatched transplant recipients. In addition, reactivity to several minor antigens has been strongly implicated in the pathogenesis of acute cellular rejection (ACR) (Giral et al., 2013), acute antibody-mediated rejection (AMR) (Yamani et al., 2006; Yamani et al., 2004), and chronic rejection (Burlingham et al., 2007; Goers et al., 2008; Nath et al., 2011; Sarma et al., 2012; Soulez et al., 2012) in multiple transplanted organs. In summary, the means to specifically target minor antigen reactivity is believed to be able to dramatically impact and improve transplant outcomes.

As used herein, “AT” stands for adoptively transferred; “DC” stands for dendritic cell; “flu” stands for influenza; “LN” stands for lymph node; “poly(I:C)” stands for polyinosinic-polycytidylic acid and “WT” stands for wild-type.

Graft versus host disease is a common complication of allogeneic transplants in which functional immune cells in the transplanted graft recognize the recipient as “foreign” and produces an immune response to the host tissue.

One embodiment of the present invention is a method of treating or preventing graft versus host disease and/or graft rejection in a subject in need thereof. The method includes administering to the subject a therapeutically effective amount of a pharmaceutical composition comprising an antibody against human antigen presenting cell (APC)-specific surface molecules.

APC is a cell that can present antigen bound to MHC class I or class II molecules to T cells. APCs include, but are not limited to dendritic cells, monocytes, macrophages, B cells, T cells and Langerhans cells. In a preferred aspect, the APC is a dendritic cell. In a preferred aspect, the APC-specific surface molecules are cluster of differentiation 103 (CD103), cluster of differentiation 8 (CD8), chemokine (C Motif) Receptor 1 (XCR1) and combinations thereof.

An antigen is a compound or composition or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond.

A “therapeutically effective amount” as used here is a quantity of composition or a cell to achieve a desired effect in a subject being treated.

Another embodiment of the present invention is a method of treating and/or preventing rejections of minor antigen-mismatched grafts in a subject in need thereof. The method comprises administering to the subject a therapeutically effective amount of a pharmaceutical composition comprising an agent capable of depleting and/or inhibiting Batf3-dependent antigen presenting cells.

Still another embodiment of the present invention is a method of depleting or inhibiting Batf3-dependent antigen-presenting cells in a subject in need thereof. The method comprises administering to the subject a pharmaceutical composition comprising an agent for inducing depletion or inhibition of Batf3-dependent cells. The agent includes, but it not limited to, an antibody, a small molecule or a peptide that is capable of binding to and depleting and/or inhibiting the Batf3-dependent antigen presenting cells. In a preferred aspect, the antibody is C-type lectin domain family 9 (Clec9a), Cluster of Differentiation 1a (CD1a), DEC205, Cluster of Differentiation 1c (CD1c) or a combination of one or more of these antibodies. In one aspect, a monoclonal specific antibody against CD1c and/or CD1a is administered to deplete CD1c and/or CD1c expressing cells.

In any of the embodiments of the invention, the method comprises administering a therapeutically effective amount of an agent capable of depleting and/or inhibiting Batf3-dependent antigen-presenting cells in a graft. In one aspect, depleting also means to kill the Batf3-dependent antigen-presenting cells.

In still another aspect, the subject is further administered a toll-like receptor 7 (TLR7) inhibitor is administered. As described in Example 8, the inventor has determined that TLR7 stimulation bypasses the requirement for Batf3-dependent DCs to reject cells expressing mismatched minor antigens by altering the phenotype of Batf3-independent DCs to acquire the intrinsic characteristics of Batf3-dependent DCs. Thus, by further administering one or more TLR7 inhibitors, this bypass may be inhibited. In one aspect, the TLR7 inhibitor is a TLR7 antagonist.

In one embodiment of the present invention, methods provide for treating a subject in need of or undergoing a transplant. For example, treatment for reducing graft rejection, promoting graft survival and promoting prolonged graft function by administering to a subject in need thereof a therapeutically effective amount of a composition as described herein.

The subject has or will receive an allograft. Such an allograft includes but is not limited to an organ, tissue or cells. In one aspect, the subject is a mammal. In a preferred aspect, the subject is a human.

Embodiments of the present invention provide for methods of promoting translation, graft survival, reducing graft rejection and/or reducing or preventing side effects associated with graft rejection. In accordance with these embodiments side effects include conditions associated with graft versus host disease or graft rejection. In one embodiment, symptoms or signs may include but are not limited to one or more the following, malaise, fever, dry cough, myalgias, chest pains, ventilatory compromise, sweating, nausea, vomiting, abdominal pain, bloody diarrhea, mucosal ulcerations, reduced renal function, reduced pulmonary function, reduced cardiac function, gastrointestinal ulceration, pulmonary failure, and skin ulceration.

Any of the embodiments disclosed herein further include one or more therapeutically effective amount of anti-microbial drugs, anti-inflammatory agent, immunomodulatory agent, immunosuppressive agent and combinations thereof. Examples of anti-rejection agents/drugs include but are not limited cyclosporine, azathioprine, corticosteroids, rapamycin, mizoribine, and combinations thereof.

In addition, other combination compositions of methods disclosed herein include additional antibody-based therapies, including but not limited to, polyclonal anti-lymphocyte antibodies, monoclonal antibodies directed at the T-cell antigen receptor complex, monoclonal antibodies directed at additional cell surface antigens, such as interleukin-2 receptor alpha.

Antibody based therapies of the present invention may be used as induction therapy and/or anti-rejection drugs.

In one aspect of the invention, the pharmaceutical composition may be administered to the subject in a manner such as subcutaneous, intravenous, intranasal, inhalation, oral, transdermal, and/or topical. The composition may be administered to the subject in an appropriate pharmaceutically acceptable carrier, including diluents such as saline and aqueous buffer solutions.

Pharmaceutical compositions suitable for injectable use may be administered by means known in the art. For example, sterile aqueous solutions (where water soluble) or dispersion and sterile powders for the extemporaneous preparation of sterile injectable solutions or dispersion by may be used. The composition can be sterile and can be fluid to the extent that it easy to use via a syringe.

Nasal solutions or sprays, aerosols or inhalants may also be used to deliver the pharmaceutical compositions.

Pharmaceutical compositions are administered in an amount, and with a frequency, that is effective to inhibit or alleviate side effects of a transplant (graft) and/or to reduce or prevent rejection.

In one aspect, the subject is administered the pharmaceutical composition concurrently with receipt of a graft or transplant. In one aspect, the subject is administered the pharmaceutical composition following receipt of a graft. In one aspect, the composition is administered to the subject immediately following the subject's receipt of the graft or transplant, within 24 hours after receipt of the graft, 24 hours after receipt of the graft or transplant, one day after, two days after, three days after, four days after, five days after, six days after, seven days, 2 weeks after, 3 weeks after, or 4 weeks after receipt of the graft or transplant. In yet another aspect, the subject is administered the pharmaceutical composition prior to receipt of a graft, such as 1 day before the transplant. The pharmaceutical composition may be administered one or more times following and/or prior to receipt of the graft or transplant. In one instance, the pharmaceutical composition is administered one or more times after receipt of the graft or transplant for an additional 1 week, 2 weeks, 3 weeks, 4 weeks, 2 months, 3 months, 4 months, 5 months, 6 months, 7 months, 8 months, 9 months, or 1 year, or until tolerance develops and overrides immunity. The pharmaceutical composition may be administered daily, every other day, biweekly, weekly, monthly. It will be apparent that, for any particular subject, specific dosage regimens may be adjusted over time according to the individual need.

Each publication or patent cited herein is incorporated herein by reference in its entirety. The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

This example provides the materials and methods used in the examples disclosed herein. Mice: C57BL/6 or Balb/c CD45.1 or CD45.2 mice aged 6-8 weeks, 129SvEv/BL6 F2 (controls for 129SvEv/BL6 Batf3^{-/-} F2 mice were provided by Dr. Kenneth Murphy), C57BL/6

Batf3^{-/-}, Balb/c Batf3^{-/-}, OT-I, OT-II, DO11.10 and CL4 mice were purchased from Jackson Laboratory or Charles River. Mice were housed in a specific pathogen-free environment at National Jewish Health, an AAALAC accredited institution, and used in accordance with protocols approved by the IACUC.

Rejection model against male-specific minor antigens: Spleen and LNs were harvested from OT-I, OT-II, DO11.10 and CL4 mice. Two million cells were adoptively transferred (AT) i.v. into mice. Next day, mice were immunized via the i.n. route with of antigen 2 μg soluble ovalbumin (OVA) or 1 μg long-flu peptide in 50 μl of PBS (Desch, A.N., et al. 2014). To observe the acceptance or rejection of AT male T cells, mice were re-challenged at day 18 with 100 μg OVA or 10 μg flu peptide (i.e. recall response). Two days after the re-challenge lung-draining LNs were examined for the presence or absence of AT male T cells.

In vivo cytotoxic T-cell assay: 2 μg soluble OVA (0.22μ filtered, Grade VII Sigma) was delivered i.n. to promote expansion of male OT-I T cells. Ten days after immunization, target cells were labeled using 10 μM CFSE. Target cells were 10′ (1:1) CFSE-labeled male CD45.2 and female CD45.1 splenocytes. 3d later, spleens were harvested and specific killing of AT congenic target cells was assessed.

Flow cytometry: Single-cell suspensions were prepared from lung-draining LNs. Cells were stained for 30 min with the following monoclonal antibodies (mAbs): Pacific Blue- or e450- or PERCP-Cy5.5- conjugated mAbs to CD8, CD4 and CD44; APC- or PE-conjugate mAb to CD45.1, CD45.2 and CD90.1; FITC-conjugated mAbs Va2 or KJ1-26 (binds the T-cell receptor expressed on DO11.10). Analysis was performed on the BD LSR II and FlowJo (Tree Star, USA).

Skin transplantation: Balb/c Batf3^{-/-} male and female skins were transplanted onto syngeneic WT and Batf3^{-/-} female mice. Full thickness donor skin was acquired from the abdominal surface. Recipient mice were treated with buprenorphine (0.03 mg/kg body weight) and anaesthetized with isoflurane. Graft beds were prepared on the left shoulder of recipient mice by excising skin equivalent to the size of the donor graft, ˜1 cm². Grafts were held in place with Vetbond tissue adhesive glue (3M), and covered with Vaseline coated gauze (Covidien) and triple antibiotic cream (Actavis Mid Atlantic). Finally, grafted area was wrapped with adhesive wrap (Fischer) and 500 μl of saline was injected subcutaneously on the recipients' flanks to aid in hydration and recovery. Skin grafts were assessed for acceptance or rejection up to 60 days post transplantation. Time of rejection was defined as the day when a necrotic donor graft had completely fallen off a recipient.

Statistics: Statistical analysis was conducted using InStat and Prism software (GraphPad). Statistical tests were performed using two-tailed Student's t-test. A value of P<0.05 was considered statistically significant.

In vivo cytotoxic T-cell assay for male skin grafts onto female mice: Thirty days after skin transplantation, target cells were labeled using 10 μM CFSE. Target cells were 10⁷ (1:1) CFSE-labeled male CD45.2 and female CD45.1 splenocytes. Three days later, spleens were harvested and specific killing of adoptively transferred congenic target cells was assessed. Statistical analysis was conducted using InStat and Prism software (GraphPad). All results are expressed as the mean±s.e.m. Statistical tests were performed using two-tailed Student's t-test. A value of P<0.05 was considered statistically significant.

### Example 2

This example shows that Batf3-dependent DCs are required for rejection of minor antigen-mismatched grafts.

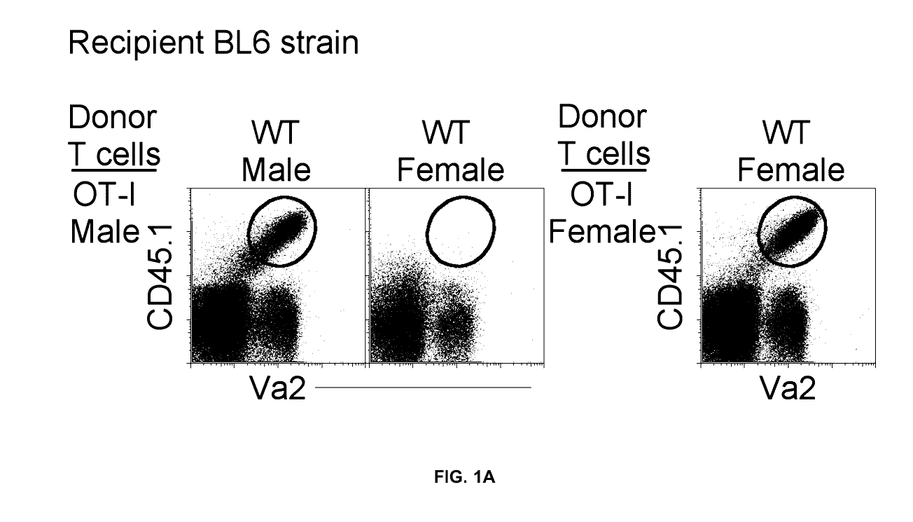

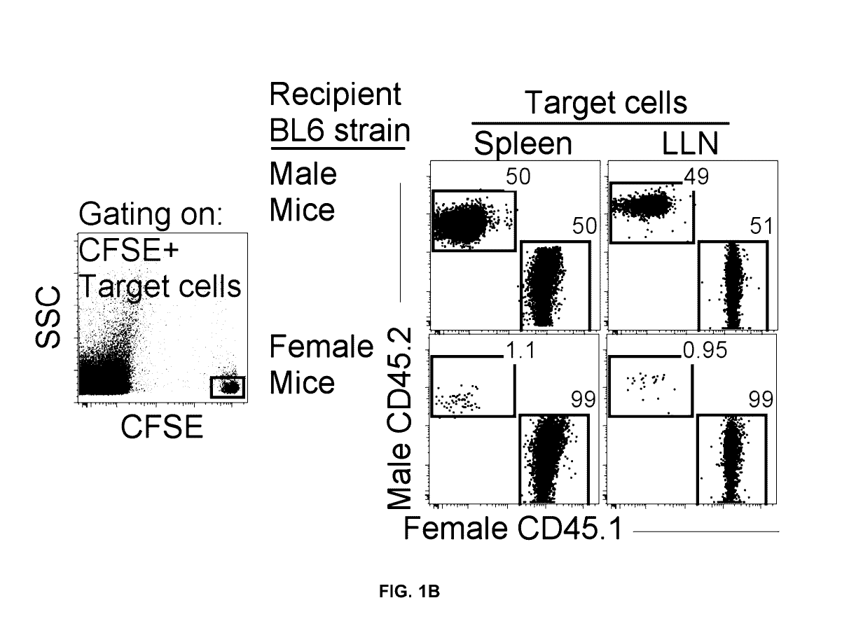

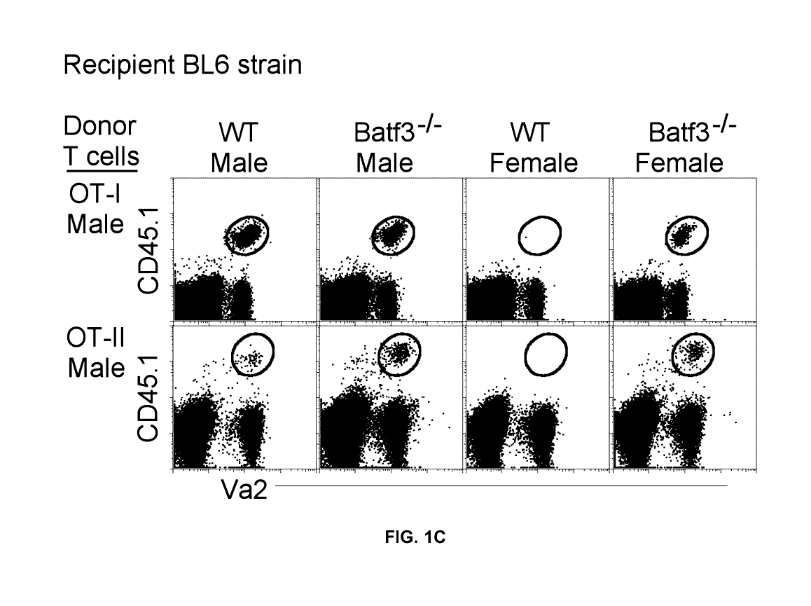

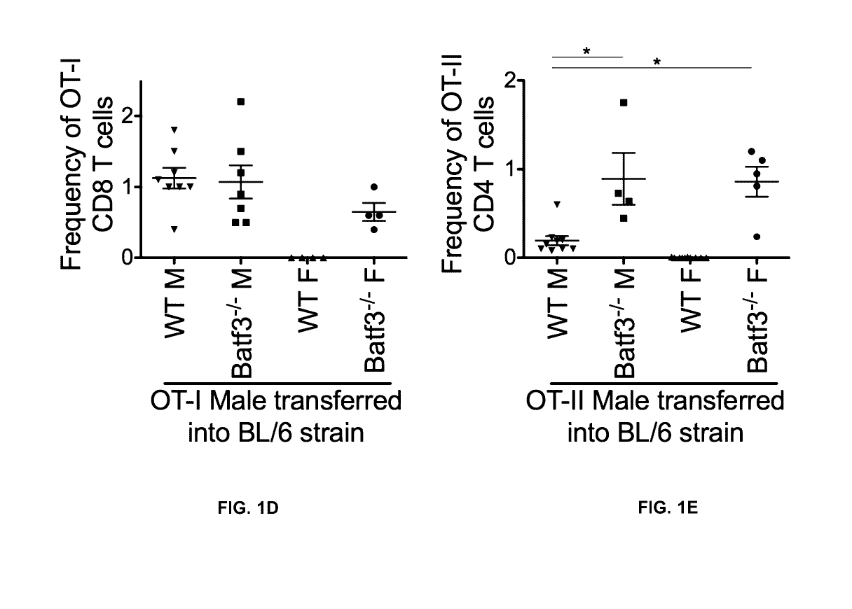

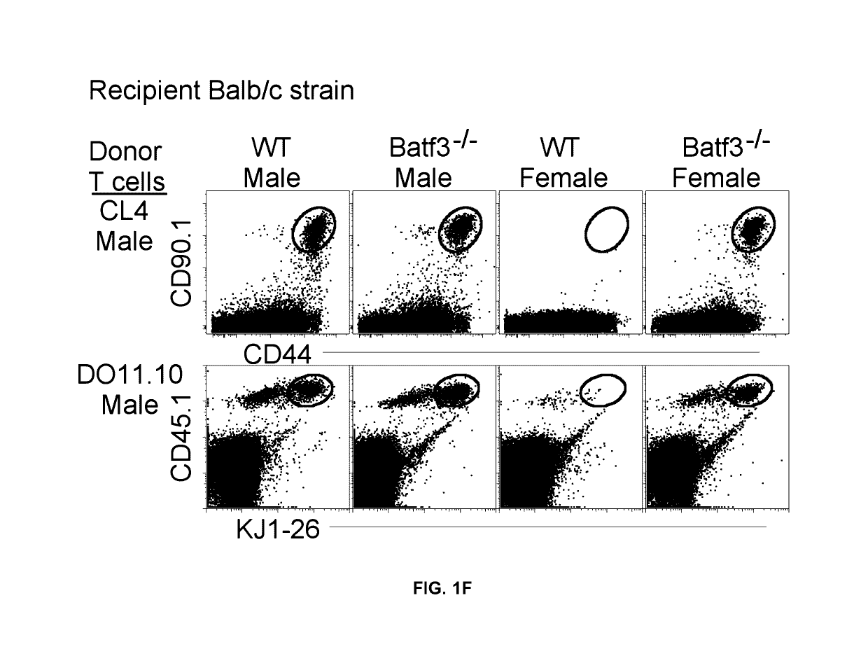

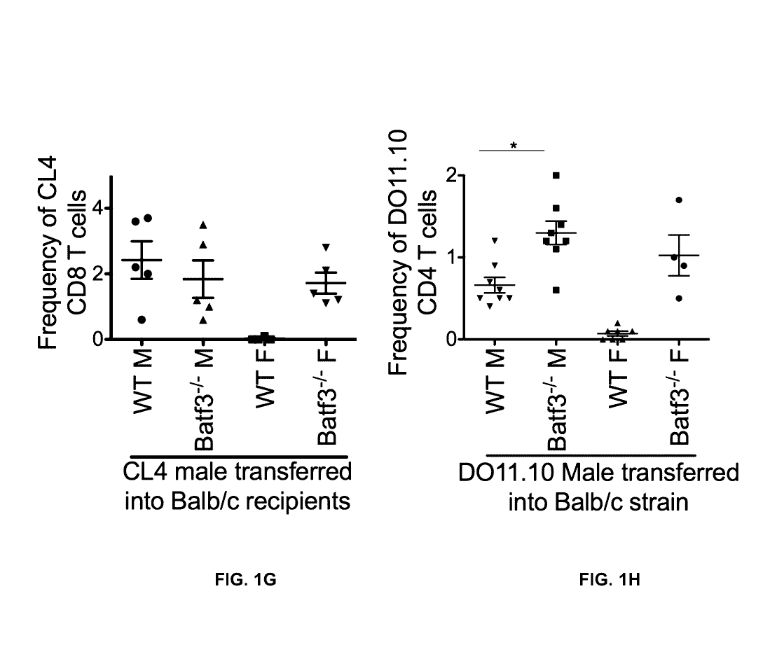

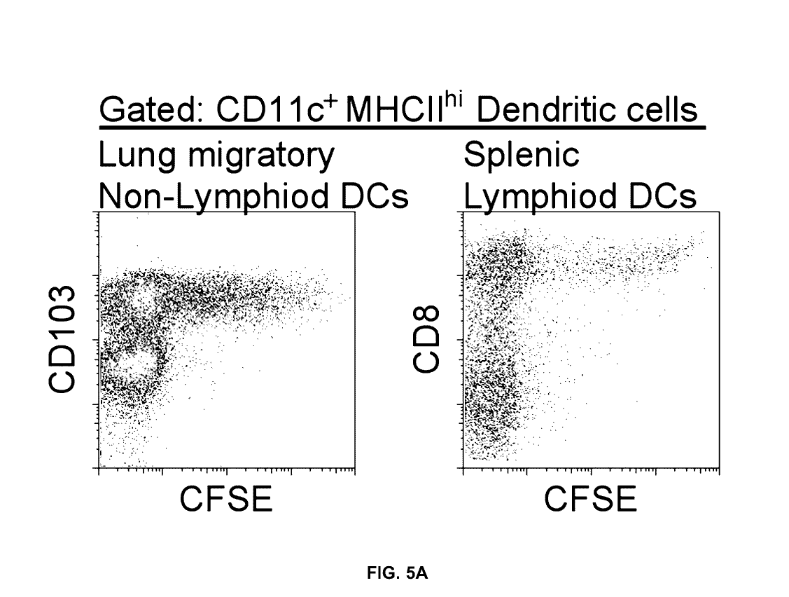

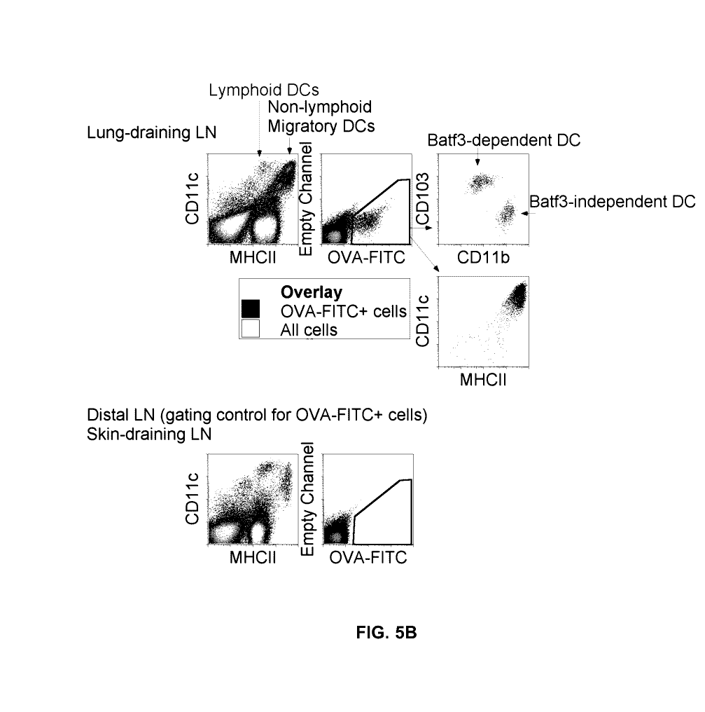

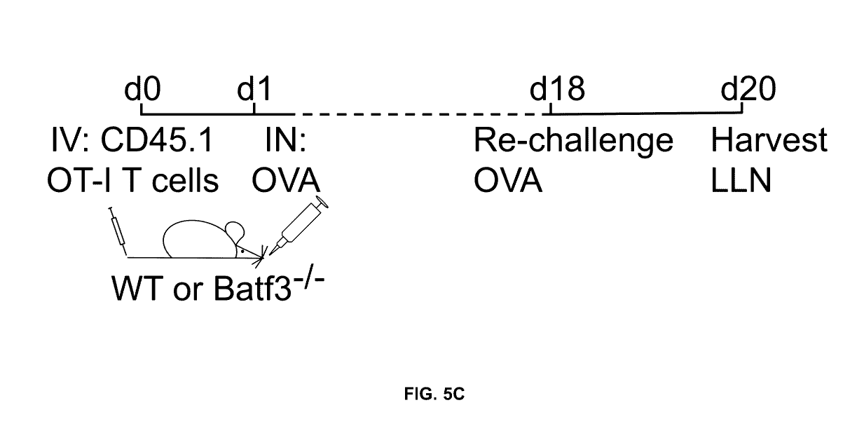

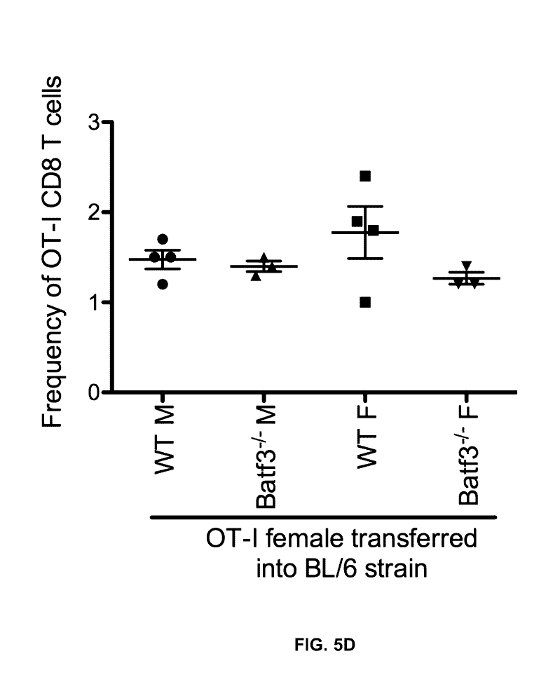

In order to demonstrate that Batf3-dependent DCs are required for rejection of minor antigen-mismatched grafts, a graft rejection system was used. Grafted male cells and tissues are rejected in MHC matched syngeneic female mice due to the development of cytotoxic T cells (CTL) against male-specific minor antigens (Goldberg, E. H., et al. 1973. Detection of H-Y (male) antigen on mouse lymph node cells by the cell to cell cytotoxicity test. Transplantation 15: 334-336; Gavin, M. A., B., et al. 1994. Major histocompatibility complex class I allele-specific peptide libraries: identification of peptides that mimic an H-Y T cell epitope. Eur J Immunol 24: 2124-2133). Therefore, the rejection of adoptively transferred (AT) male lymphocytes into syngeneic WT female mice was examined. Male CD45.1 OVA-specific CD8⁺ T cells (OT-I T cells) were intravenously injected into syngeneic CD45.2 WT male and female mice. One day after AT, the mice were immunized via the intranasal route with 2 μg of soluble OVA. Delivery and trafficking of the antigen by two migratory DCs, Batf3-dependent and Batf3-independent DCs (FIG. 5B) (Jakubzick, C., et al. 2008. Optimization of methods to study pulmonary dendritic cell migration reveals distinct capacities of DC subsets to acquire soluble versus particulate antigen. Journal of immunological methods 337: 121-131), initiated expansion of the male OT-I T cells in the lung-draining LNs. To observe the acceptance or rejection of male OT-I T cells, mice were re-challenged at day 18 with 100 μg OVA (i.e. recall response). Two days after the re-challenge, the lung-draining LNs were examined for the presence or absence of AT male OT-I T cells (schematic diagram, FIG. 5C). As expected, there was no recall of the AT CD45.1 male OT-I T cells (i.e. the cells were rejected) in syngeneic WT female mice compared to syngeneic WT male mice (FIG. 1A). The rejection of male OT-I T cells in syngeneic WT female mice was not due to other mismatched minor antigens potentially present in the OT-I mouse strain, as AT female OT-I T cells were not rejected in syngeneic WT female mice (FIG. 1A).

### Example 3

This example confirms that the absence of male cells in female mice post the recall response was due to the induction of endogenous male antigen-specific CTLs. An in vivo CTL killing assay was used (Gavin, M. A., et al.; Tyznik, A. J., and M. J. Bevan. 2007. The surprising kinetics of the T cell response to live antigenic cells. Journal of immunology 179: 4988-4995).

Ten days after the AT of male OT-I T cells and administration of the OVA antigen, CFSE-labeled target cells, WT CD45.2 male and CD45.1 female splenocytes, were intravenously delivered at a 1:1 ratio for the measurement of male antigen-specific killing. Three days later, in vivo CTL responses were assessed by measuring the killing of male target cells compared to female target cells (FIG. 1B). As expected, WT female mice that received male OT-I T cells displayed almost complete killing of male target cells compared to WT male mice where no killing was observed (FIG. 1B). This experiment concurs with the well-established finding that cells expressing male antigen are rejected in female mice via the induction of endogenous male antigen-specific CTL (Tyznik, A. J., 2007).

### Example 4

In order to determine whether Batf3-dependent DCs promote the rejection of male-specific minor antigens in female mice, the inventor next examined whether Batf3^{-/-} female mice (selectively lacking Batf3-dependent DCs) (Hildner, K., et al.; Desch, A. N., et al.; and Edelson, B. T., et al.) reject AT male OT-I T cells as observed for WT female mice. However, after re-challenge with antigen, male OT-I T cells were not rejected in Batf3^{-/-} female mice, i.e the recall response was retained (FIG. 1E). Control experiment demonstrated that there was no rejection against female OT-I T cells AT into WT and Batf3^{-/-} male and female mice (FIG. 5F-5H). Furthermore, similar to the findings for male antigen-specific CD8⁺ T cells, AT male antigen-specific CD4⁺ T cells (CD45.1 OT-II T cells) were not rejected in Batf3^{-/-} female mice but were rejected in WT female mice (FIG. 1E).

### Example 5

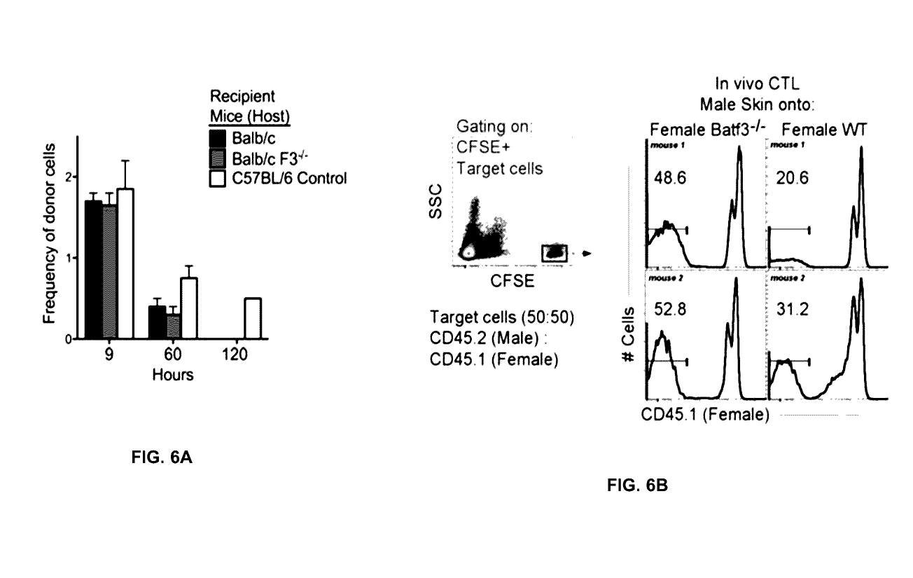

Since C57BL/6 female Batf3^{-/-} mice lacked the ability to reject male-specific minor antigens, the inventor determined whether this finding was independent of mouse strain. Balb/c Batf3^{-/-} mice were examined using the same experimental design as FIG. 1A. However, instead of C57BL/6 antigen-specific T cells, Balb/c Flu-specific CD8⁺ T cells (CL4) or OVA-specific CD4⁺ T cells (DO11.10) were AT into syngeneic WT or Batf3^{-/-} male and female mice. As anticipated, male AT antigen-specific T cells were rejected in WT female Balb/c mice but not in WT or Batf3^{-/-} male mice (FIG. 1F-1H), and in contrast to WT female mice, Batf3^{-/-} female mice did not reject the male antigen-specific T cells. To note, in an allogeneic setting with major antigen-mismatch, rejection of AT allogeneic cells occurred in both WT and Batf3^{-/-} mice (FIG. 6A). Overall, the data demonstrate that in both C57BL/6 and BALB/c female mouse strains, Batf3-dependent DCs are required for the rejection of minor antigen-mismatched grafts.

Another interesting finding with the AT CD4⁺ T cell experiments was the enhanced recall of CD4⁺ T cells in Batf3^{-/-} mice compared to WT mice (FIG. 1F-1H). This finding is supported by an elegant study by Kim, TS et. al., (2014. Distinct dendritic cell subsets dictate the fate decision between effector and memory CD8(+) T cell differentiation by a CD24-dependent mechanism. Immunity 40: 400-413) demonstrating that Batf3-independent DC (DC that preferentially presents antigen to CD4 T cells) substantially contributes to the development of memory T cells compared to Batf3-dependent DC; hence resulting in a greater recall of CD4⁺ T cells in Batf3^{-/-} mice compared to WT mice.

### Example 6

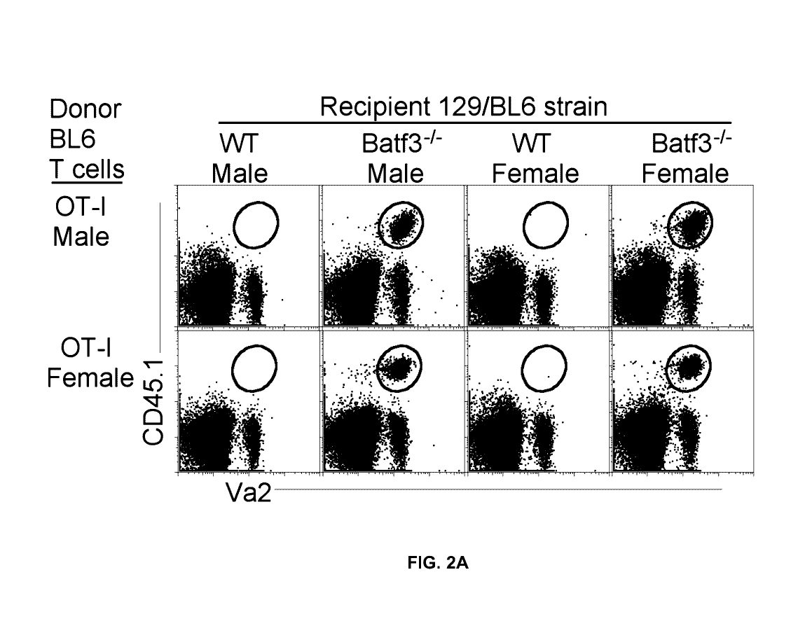

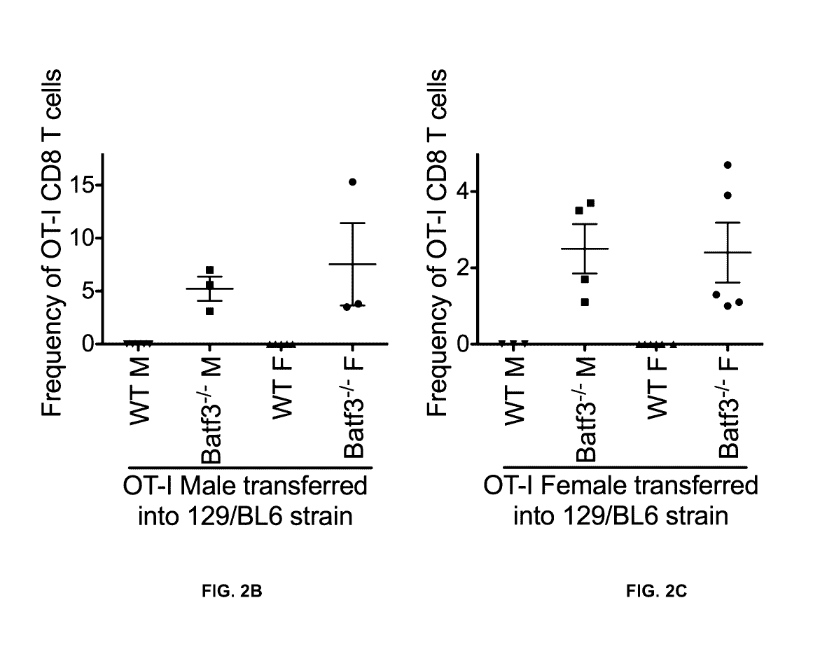

This example demonstrates that Batf3-dependent DCs are required for the rejection of AT cells expressing mismatched male-specific and/or male-nonspecific minor antigens. To assess the requirement of Batf3-dependent DCs for the rejection of minor antigen-mismatched grafts, the rejection of minor antigens in F₂ 129SvEv/BL6 Batf3^{-/-} mice (provided by Dr. Kenneth Murphy) and F₂ 129SvEv/BL6 WT mice (provided by Jackson Laboratory) was examined, which were then continuously bred to each other. In this setting, although donor C57BL/6 OT-I T cells express the same MHC molecules as recipient 129SvEv/BL6 mice, they have multiple mismatched minor histocompatibility antigens that are distinct from the male-specific minor antigens (Malarkannan, S., T,e t al. 2000. Differences that matter: major cytotoxic T cell-stimulating minor histocompatibility antigens. Immunity 13: 333-344; Simpson, E. M., et al. 1997. Genetic variation among 129 substrains and its importance for targeted mutagenesis in mice. Nat Genet 16: 19-27). Using the experimental design from FIG. 1A, the inventor adoptively transferred male C57BL/6 OT-I T cells and then observed rejection in both 129SvEv/BL6 WT male and female mice but not in 129SvEv/BL6 Batf3^{-/-} male or female mice (FIG. 2A-2C). The difference in the rejection pattern observed for AT C57BL/6 OT-I male cells into 129SvEv/BL6 male mice compared to C57BL/6 male mice (FIG. 1C-1E) was due to the mismatch of minor antigens outside of the H-Y locus. As AT C57BL/6 OT-I female cells were rejected in 129SvEv/BL6 WT male and female mice but not in 129SvEv/BL6 Batf3^{-/-} male or female mice (FIG. 2A-2C).

### Example 7

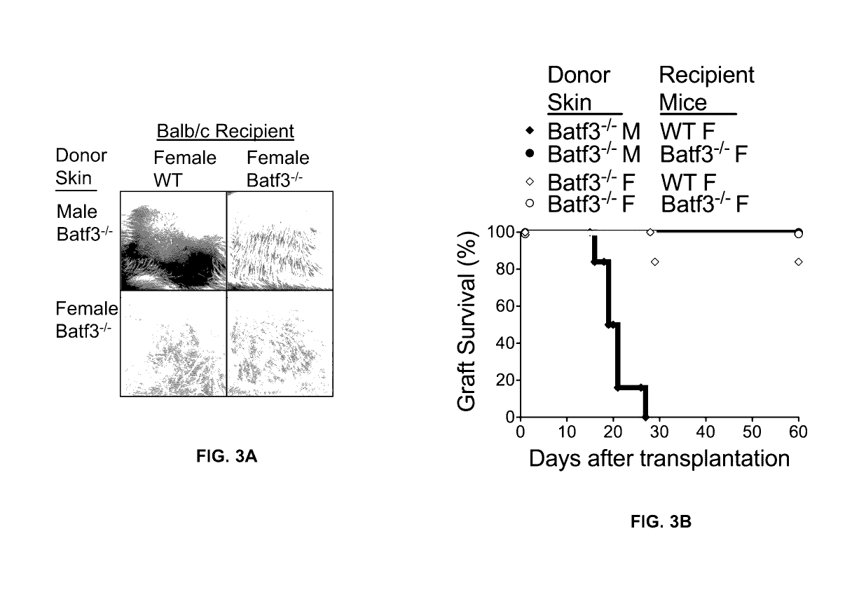

This example demonstrates that in a skin transplant model, AT T cells clearly demonstrate that Batf3-dependent DCs are required for rejection of minor antigen-mismatched grafts. Using a skin transplant model as described (Gordon, R. D., et al. 1976. The effect of allogeneic presensitization on H-Y graft survival and in vitro cell-mediated responses to H-y antigen. The Journal of experimental medicine 144: 810-820; Ashman, R. B. 1983. Primary immune responses to H-Y in BALB/c-H-2k mice. Immunogenetics 18: 125-129), the requirement of Batf3-dependent DCs in skin graft rejection was assessed. Balb/C Batf3^{-/-} male or female skin was transplanted onto syngeneic Balb/C WT female or Batf3^{-/-} female mice. Batf3^{-/-} skin was used to assure the absence of Batf3-dependent DCs in the skin transplant. As expected, male skin grafts were completely rejected by syngeneic WT female mice by 30 days post-transplantation (FIG. 3A-3B). In striking contrast, male skin was completely accepted by Batf3^{-/-} female mice (FIG. 3A-3B), and confirmed the absence of male antigen-specific CTL compared to WT female recipients (FIG. 6B). Control mice demonstrated that transplanted female skin onto syngeneic WT and Batf3^{-/-} female mice was accepted by 84% in WT mice and 100% in Batf3^{-/-} mice.

### Example 8

This example shows that TLR7 stimulation bypasses the requirement for Batf3-dependent

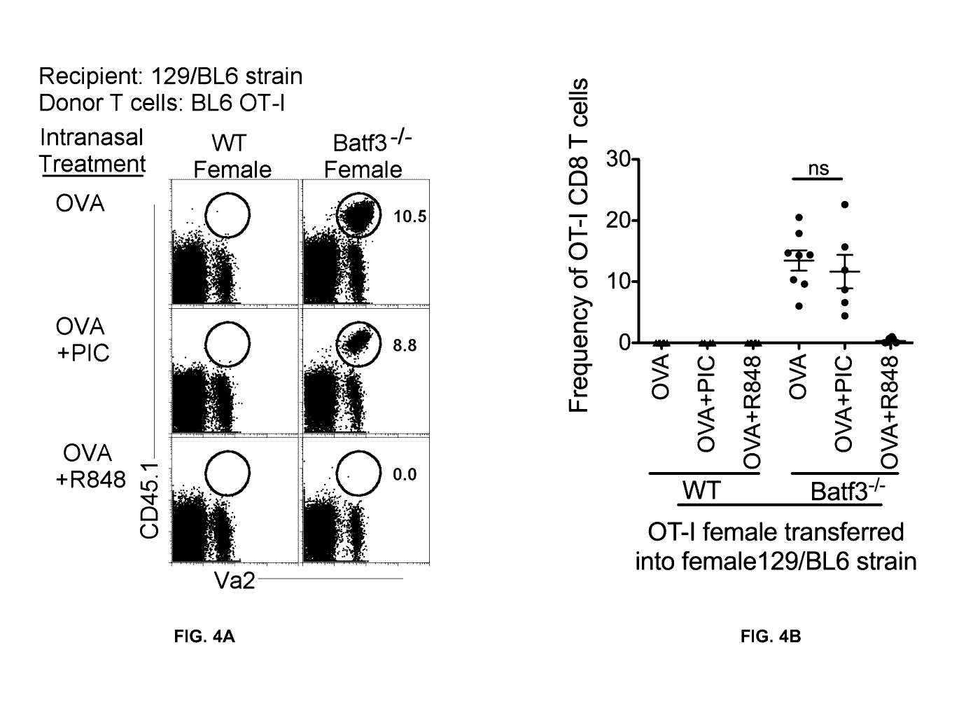

DCs to reject cells expressing mismatched minor antigens by altering the phenotype of Batf3-independent DCs to acquire the intrinsic characteristics of Batf3-dependent DCs. Batf3-independent DCs can acquire cross-presenting and cross-priming capabilities similar to Batf3-dependent DCs if the Batf3-independent DCs are stimulated with a Toll-like receptor 7 (TLR7) ligand (single-stranded RNA, R848) but not TLR3 ligand (double-stranded RNA, Poly I:C, which selectively activates Batf3-dependent CD103⁺ DCs in tissue) (Desch, A. N., et al. 2014). Therefore, it was determined whether TLR7 stimulated Batf3-independent DCs could promote rejection of minor antigen-mismatched grafts. Consistent with those previous findings, the inventor determined that when 129SvEv/BL6 Batf3^{-/-} female mice were challenged with OVA in the presence or absence of Poly I:C, rejection of C57BL/6 OT-I female cells did not occur (FIG. 4A-4B). However, in the presence of the TLR7 ligand, R848, complete rejection of AT C57BL/6 OT-I female cells was now observed in 129SvEv/BL6 Batf3^{-/-} mice where Batf3-dependent DCs are absent (FIG. 4A-4B). Control mice, 129SvEv/BL6 WT female mice, showed complete rejection of C57BL/6 OT-I female cells (FIG. 2A-2C and FIG. 4A-4B). To note, single delivery of antigen with Poly I:C or R848 does not promote the development of Batf3-dependent DCs in Batf3^{-/-} mice (Desch, A. N., et al. 2014). Thus, TLR7 stimulation bypasses the requirement for Batf3-dependent DCs to reject cells expressing mismatched minor antigens by altering the phenotype of Batf3-independent DCs to acquire the intrinsic characteristics of Batf3-dependent DCs.

Overall these findings identify Batf3-dependent DCs as the DC subtype required for rejection of minor antigen-mismatched grafts, in the absence of concurrent TLR7 stimulation. These findings also indicate potential consequences of single-stranded RNA viruses that should be considered if a therapeutic strategy were developed to eliminate Batf3-dependent DCs during transplantation, such as with the use of a depleting antibody such as Clec9a or DEC205.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

### REFERENCES

1. Hildner, K., B. T. Edelson, W. E. Purtha, M. Diamond, H. Matsushita, M. Kohyama, B. Calderon, B. U. Schraml, E. R. Unanue, M. S. Diamond, R. D. Schreiber, T. L. Murphy, and K. M. Murphy. 2008. Batf3 deficiency reveals a critical role for CD8alpha+ dendritic cells in cytotoxic T cell immunity. Science 322: 1097-1100.
2. Sung, S. S., S. M. Fu, C. E. Rose, Jr., F. Gaskin, S. T. Ju, and S. R. Beaty. 2006. A major lung CD103 (alphaE)-beta7 integrin-positive epithelial dendritic cell population expressing Langerin and tight junction proteins. Journal of immunology 176: 2161-2172.
3. Vremec, D., M. Zorbas, R. Scollay, D. J. Saunders, C. F. Ardavin, L. Wu, and K. Shortman. 1992. The surface phenotype of dendritic cells purified from mouse thymus and spleen: investigation of the CD8 expression by a subpopulation of dendritic cells. The Journal of experimental medicine 176: 47-58.
4. Desch, A. N., G. J. Randolph, K. Murphy, E. L. Gautier, R. M. Kedl, M. H. Lahoud, I. Caminschi, K. Shortman, P. M. Henson, and C. V. Jakubzick. 2011. CD103+ pulmonary dendritic cells preferentially acquire and present apoptotic cell-associated antigen. The Journal of experimental medicine 208: 1789-1797.
5. Plantinga, M., H. Hammad, and B. N. Lambrecht. 2010. Origin and functional specializations of DC subsets in the lung. Eur J Immunol 40: 2112-2118.
6. Kim, T. S., and T. J. Braciale. 2009. Respiratory dendritic cell subsets differ in their capacity to support the induction of virus-specific cytotoxic CD8+ T cell responses. PloS one 4: e4204.
7. Tamura, T., P. Tailor, K. Yamaoka, H. J. Kong, H. Tsujimura, J. J. O'Shea, H. Singh, and K. Ozato. 2005. IFN regulatory factor-4 and -8 govern dendritic cell subset development and their functional diversity. Journal of immunology 174: 2573-2581.
8. Quantz, M. A., L. E. Bennett, D. M. Meyer, and R. J. Novick. 2000. Does human leukocyte antigen matching influence the outcome of lung transplantation? An analysis of 3,549 lung transplantations. The Journal of heart and lung transplantation: the official publication of the International Society for Heart Transplantation 19: 473-479.
9. Contreras, V., C. Urien, R. Guiton, Y. Alexandre, T. P. Vu Manh, T. Andrieu, K. Crozat, L. Jouneau, N. Bertho, M. Epardaud, J. Hope, A. Savina, S. Amigorena, M. Bonneau, M. Dalod, and I. Schwartz-Cornil. 2010. Existence of CD8alpha-like dendritic cells with a conserved functional specialization and a common molecular signature in distant mammalian species. Journal of immunology 185: 3313-3325.
10. Iyoda, T., S. Shimoyama, K. Liu, Y. Omatsu, Y. Akiyama, Y. Maeda, K. Takahara, R. M. Steinman, and K. Inaba. 2002. The CD8+ dendritic cell subset selectively endocytoses dying cells in culture and in vivo. The Journal of experimental medicine 195: 1289-1302.
11. Ferguson, T. A., J. Herndon, B. Elzey, T. S. Griffith, S. Schoenberger, and D. R. Green. 2002. Uptake of apoptotic antigen-coupled cells by lymphoid dendritic cells and cross-priming of CD8(+) T cells produce active immune unresponsiveness. Journal of immunology 168: 5589-5595.
12. den Haan, J. M., S. M. Lehar, and M. J. Bevan. 2000. CD8(+) but not CD8(-) dendritic cells cross-prime cytotoxic T cells in vivo. The Journal of experimental medicine 192: 1685-1696.
13. Kurts, C., B. W. Robinson, and P. A. Knolle. 2010. Cross-priming in health and disease. Nature reviews. Immunology 10: 403-414.
14. Desch, A. N., S. L. Gibbings, E. T. Clambey, W. J. Janssen, J. E. Slansky, R. M. Kedl, P. M. Henson, and C. Jakubzick. 2014. Dendritic cell subsets require cis-activation for cytotoxic CD8 T-cell induction. Nature communications 5: 4674.
15. Edelson, B. T., W. Kc, R. Juang, M. Kohyama, L. A. Benoit, P. A. Klekotka, C. Moon, J. C. Albring, W. Ise, D. G. Michael, D. Bhattacharya, T. S. Stappenbeck, M. J. Holtzman, S. S. Sung, T. L. Murphy, K. Hildner, and K. M. Murphy. 2010. Peripheral CD103+ dendritic cells form a unified subset developmentally related to CD8alpha+ conventional dendritic cells. The Journal of experimental medicine 207: 823-836.
16. Bedoui, S., P. G. Whitney, J. Waithman, L. Eidsmo, L. Wakim, I. Caminschi, R. S. Allan, M. Wojtasiak, K. Shortman, F. R. Carbone, A. G. Brooks, and W. R. Heath. 2009. Cross-presentation of viral and self antigens by skin-derived CD103+ dendritic cells. Nature immunology 10: 488-495.
17. GeurtsvanKessel, C. H., M. A. Willart, L. S. van Rijt, F. Muskens, M. Kool, C. Baas, K. Thielemans, C. Bennett, B. E. Clausen, H. C. Hoogsteden, A. D. Osterhaus, G. F. Rimmelzwaan, and B. N. Lambrecht. 2008. Clearance of influenza virus from the lung depends on migratory langerin+CD11b− but not plasmacytoid dendritic cells. The Journal of experimental medicine 205: 1621-1634.
18. Ferris, S. T., J. A. Carrero, J. F. Mohan, B. Calderon, K. M. Murphy, and E. R. Unanue. 2014. A minor subset of Batf3-dependent antigen-presenting cells in islets of Langerhans is essential for the development of autoimmune diabetes. Immunity 41: 657-669.
19. Goldberg, E. H., F. W. Shen, and S. Tokuda. 1973. Detection of H-Y (male) antigen on mouse lymph node cells by the cell to cell cytotoxicity test. Transplantation 15: 334-336.
20. Gavin, M. A., B. Dere, A. G. Grandea, 3rd, K. A. Hogquist, and M. J. Bevan. 1994. Major histocompatibility complex class I allele-specific peptide libraries: identification of peptides that mimic an H-Y T cell epitope. Eur J Immunol 24: 2124-2133.
21. Jakubzick, C., J. Helft, T. J. Kaplan, and G. J. Randolph. 2008. Optimization of methods to study pulmonary dendritic cell migration reveals distinct capacities of DC subsets to acquire soluble versus particulate antigen. Journal of immunological methods 337: 121-131.
22. Tyznik, A. J., and M. J. Bevan. 2007. The surprising kinetics of the T cell response to live antigenic cells. Journal of immunology 179: 4988-4995.
23. Kim, T. S., S. A. Gorski, S. Hahn, K. M. Murphy, and T. J. Braciale. 2014. Distinct dendritic cell subsets dictate the fate decision between effector and memory CD8(+) T cell differentiation by a CD24-dependent mechanism. Immunity 40: 400-413.
24. Malarkannan, S., T. Horng, P. Eden, F. Gonzalez, P. Shih, N. Brouwenstijn, H. Klinge, G. Christianson, D. Roopenian, and N. Shastri. 2000. Differences that matter: major cytotoxic T cell-stimulating minor histocompatibility antigens. Immunity 13: 333-344.
25. Simpson, E. M., C. C. Linder, E. E. Sargent, M. T. Davisson, L. E. Mobraaten, and J. J. Sharp. 1997. Genetic variation among 129 substrains and its importance for targeted mutagenesis in mice. Nat Genet 16: 19-27.
26. Gordon, R. D., B. J. Mathieson, L. E. Samelson, E. A. Boyse, and E. Simpson. 1976. The effect of allogeneic presensitization on H-Y graft survival and in vitro cell-mediated responses to H-y antigen. The Journal of experimental medicine 144: 810-820.
27. Ashman, R. B. 1983. Primary immune responses to H-Y in BALB/c-H-2k mice. Immunogenetics 18: 125-129.

What is claimed:

1. A method of treating or preventing graft versus host disease in a subject in need thereof, comprising administering to the subject a therapeutically effective amount of a pharmaceutical composition comprising an antibody against human antigen presenting cell (APC)-specific surface molecules, wherein the APC is a dendritic cell.

2. The method of claim 1, wherein the subject has or will receive an allograft.

3. The method of claim 2, wherein the subject is an MHC-matched individual to the allograft.

4. The method of claim 2, wherein the allograft is an organ, tissue or cells.

5. The method of claim 1, wherein the pharmaceutical composition is administered concurrently, following or prior to a transplant of a graft.

6. The method of claim 1, wherein the APC-specific surface molecules are selected from the group consisting of cluster of differentiation 103 (CD 103), cluster of differentiation 8 (CD8), chemokine (C Motif) Receptor 1 (XCR1) and combinations thereof.

7. The method of claim 1, wherein the antibody depletes or inhibits Batf3-dependent antigen presenting cells.

8. The method of claim 7, wherein the antibody is selected from the group consisting of C-type lectin domain family 9 (Clec9a), Cluster of Differentiation 1a (CD1a), DEC205, Cluster of Differentiation 1c (CD1c) and combinations thereof.

9. The method of claim 1, further comprising administering a toll-like receptor 7 (TLR7) inhibitor to the subject.

10. A method of treating or preventing rejection of minor antigen-mismatched grafts in a subject in need thereof, comprising administering to the subject a therapeutically effective amount of a pharmaceutical composition comprising an agent capable of depleting or inhibiting Batf3-dependent antigen presenting cells.

11. The method of claim 10, wherein the subject has or will receive an allograft.

12. The method of claim 11, wherein the allograft is an organ, tissue or cells.

13. The method of claim 10, wherein the pharmaceutical composition is administered concurrently, following or prior to a transplant of a graft.

14. The method of claim 10, wherein the agent is an antibody.

15. The method of claim 14, wherein the antibody is selected from the group consisting of C-type lectin domain family 9 (Clec9a), Cluster of Differentiation 1a (CD1a), DEC205, Cluster of Differentiation 1c (CD1c) and combinations thereof

16. The method of claim 10, further comprising administering a toll-like receptor 7 (TLR7) inhibitor to the subject.

17. A method of depleting or inhibiting Batf3-dependent antigen-presenting cells in a subject in need thereof comprising administering to the subject a pharmaceutical composition comprising an agent for inducing depletion or inhibition of Batf3-dependent cells.

18. The method of claim 17, wherein the agent binds to Batf3-dependent cells.

19. The method of claim 17, wherein the agent is an antibody selected from the group consisting of C-type lectin domain family 9 (Clec9a), Cluster of Differentiation 1a (CD1a), DEC205, Cluster of Differentiation 1c (CD1c) and combinations thereof.

20. The method of claim 17, wherein the subject is a transplant recipient.
